(19) United States
(12) Patent Application Publication
(10) Pub. No.: US 2006/0025482 A1
(43) Pub. Date: Feb. 2, 2006

(54) (-)-Hydroxycitric acid for the modulation of angiotensin-converting enzyme

(76) Inventor: Dallas L. Clouatre, Santa Monica, CA (US)

Correspondence Address: DALLAS L. CLOUATRE, 1223 WILSHIRE BLVD #761, SANTA MONICA, CA, 90403-5400, US

(21) Appl. No.: 11/152,698

(22) Filed: Jun. 14, 2005

Related U.S. Application Data
(60) Provisional application No. 60/599,223, filed on Jul. 29, 2004.

(51) Int. Cl. A61K 31/19

(52) U.S. Cl. 514/574

(57) ABSTRACT

The invention teaches that supplementation with (−)-hydroxycitrate constitutes a novel means of modulating the angiotensin-converting enzyme (ACE)/renin-angiotensin-aldosterone system and is useful for preventing, treating and ameliorating conditions involving the angiotensin-converting enzyme (ACE)/renin-angiotensin-aldosterone system. The discovery that HCA has angiotensin-converting enzyme (ACE)/renin-angiotensin-aldosterone system-moderating effects allows for the creation of novel and more efficacious approaches to preventing and ameliorating conditions that arise from excessive ACE activity. These include cardiovascular diseases in general, heart failure, ventricular remodeling, ejection fraction issues, atrial fibrillation, and a wide variety of renal conditions. Other health conditions discovered to be influenced by the angiotensin-converting enzyme (ACE)/renin-angiotensin-aldosterone system would similarly be expected to be influenced. It is yet a further advantage of the present invention to provide a means—one that is accompanied by few or no side effects—of maintaining such improved status without resort to special diets. Furthermore, this discovery makes possible the development of adjuvant modalities that can be used to improve the results realized with other treatment compounds while at the same time reducing the side effects normally found with such drugs. HCA delivered in the form of its potassium salt is efficacious at a daily dosage (bid or tid) of between 750 mg and 10 grams, preferably at a dosage of between 3 and 6 grams for most individuals. A daily dosage above 10 grams might prove desirable under some circumstances, such as with extremely large or resistant individuals, but this level of intake is not deemed necessary under normal conditions.

### PROVISIONAL PATENT APPLICATION FILING

Entitled to the benefit of Provisional Patent Application Ser. No. 60/599223 filed Jul. 29, 2004, “(−)-Hydroxycitric Acid for the Modulation of Agiotension-Converting Enzyme.”

### BACKGROUND OF THE INVENTION

1. Field Of The Invention

This invention relates to pharmaceutical compositions containing (−)-hydroxycitric acid, its salts and related compounds useful for modulating the metabolism of angiotensin-converting enzyme (ACE) and the renin-angiotensin-aldosterone system.

2. Description Of Prior Art

(−)-Hydroxycitric acid (abbreviated herein as HCA), a naturally-occurring substance found chiefly in fruits of the species of Garcinia, and several synthetic derivatives of citric acid have been investigated extensively in regard to their ability to inhibit the production of fatty acids from carbohydrates, to suppress appetite, and to inhibit weight gain. (Sullivan A C, Triscari J. Metabolic regulation as a control for lipid disorders. I. Influence of (−)-hydroxycitrrate on experimentally induced obesity in the rodent. American Journal of Clinical Nutrition 1977;30:767-775.) Weight loss benefits were first ascribed to HCA, its salts and its lactone in U.S. Pat. No. 3,764,692 granted to John M. Lowenstein in 1973. The claimed mechanisms of action for HCA, most of which were originally put forth by researchers at the pharmaceutical firm of Hoffmann-La Roche, have been summarized in at least two United States Patents. In U.S. Pat. No. 5,626,849 these mechanisms are given as follows: “(−) HCA reduces the conversion of carbohydrate calories into fats. It does this by inhibiting the actions of ATP-citrate lyase, the enzyme that converts citrate into fatty acids and cholesterol in the primary pathway of fat synthesis in the body. The actions of (−) HCA increase the production and storage of glycogen (which is found in the liver, small intestine and muscles of mammals) while reducing both appetite and weight gain. (−) Hydroxycitric acid also causes calories to be burned in an energy cycle similar to thermogenesis . . . (−) HCA also increases the clearance of LDL cholesterol . . . .” U.S. Pat. No. 5,783,603 further argues that HCA serves to disinhibit the metabolic breakdown and oxidation of stored fat for fuel via its effects upon the compound malonyl CoA and that gluconeogenesis takes place as a result of this action. The position that HCA acts to unleash fatty acid oxidation by negating the effects of malonyl CoA with gluconeogenesis as a consequence (McCarty M F. Promotion of hepatic lipid oxidation and gluconeogenesis as a strategy for appetite control. Medical Hypotheses 1994;42:215-225) is maintained in U.S. Pat. No. 5,914,326.

Almost all the primary research performed on HCA was carried out by Hoffman-La Roche nearly three decades ago. The conclusion of the Roche researchers was that “no significant differences in plasma levels of glucose, insulin, or free fatty acids were detected in (−)-hydroxycitrate-treated rats relative to controls. These data suggest that peripheral metabolism, defined in the present context as metabolite flux, may be involved in appetite regulation . . .” (Sullivan, Ann C. and Joseph Triscari. Possible interrelationhip between metabolite flux and appetite. In D. Novin, W. Wyriwicka and G. Bray, eds., Hunger: Basic Mechanisms and Clinical Implications (New York: Raven Press,1976) 115-125.)

HCA is highly researched as of 2005, with 157 citations appearing on PubMed/Medline under “hydroxycitrate” and 101 appearing under “hydroxycitric acid.” Quite surprisingly, until now it has not been realized that the compound influences the renin-angiotensin-aldosterone system. HCA has been discovered by the inventor to modulate the metabolism of angiotensin-converting enzyme (ACE) and the the renin-angiotensin-aldosterone system. ACE directly and indirectly influences a wide variety of physiologic mechanisms governing cardiovascular and renal functions.

For instance, activation of the renin-angiotensin-aldosterone and adrenergic nervous systems plays a major role in the progression of heart failure, and inhibitors and antagonists of these neurohormonal systems improve outcomes. Angiotensin-converting enzyme inhibitors and aldosterone antagonists have been shown to improve parameters such as ventricular remodeling, ejection fraction, and renal function and to reduce rates of morbidity and mortality. (Hiestand B, Abraham W T. Implications of heart failure drug trials: COMET, CHARM, EPHESUS. Rev Cardiovasc Med. 2005;6 Suppl 2:S4-11.) Another use of angiotensin-converting enzyme inhibitors is for the prevention of atrial fibrillation. (Healey J S, Baranchuk A, Crystal E, Morillo C A, Garfinkle M, Yusuf S, Connolly S J. Prevention of atrial fibrillation with Angiotensin-converting enzyme inhibitors and Angiotensin receptor blockers a meta-analysis. J Am Coll Cardiol. 2005 June 7;45(11):1832-9.) Effects upon kidney health can be substantial, such as in end stage kidney failure. (Chiurchiu C, Remuzzi G, Ruggenenti P. Angiotensin-converting enzyme inhibition and renal protection in nondiabetic patients: the data of the meta-analyses. J Am Soc Nephrol. 2005 March; 16 Suppl 1:S58-63.)

Many of these recognized cardiovascular and renal benefits are separable and distinct from one of the classic uses of ACE inhibitors, which is for reducing hypertension (an employment for which see our pending patent utilizing HCA). Unfortunately, current pharmaceutical ACE inhibitors can have side effects., such as hyperkalaemia, under certain circumstances. Some patients also are intolerant of ACE inhibitors due to the development of a chronic persistent low-level cough. Hence, there remains a need for new ACE inhibitors that provide expected benefits while avoiding known issues. HCA fills this void. No existing literature teaches such a role for HCA despite more than three decades of active research on the compound. The inventor's discovery that HCA is an ACE inhibitor thus clearly is novel.

### SUMMARY OF THE INVENTION

The inventor has discovered that supplementation with (−)-hydroxycitric acid, its salts and related compounds is useful for modulating the metabolism of angiotensin-converting enzyme (ACE) and the renin-angiotensin-aldosterone system. The resulting benefits of HCA are especially pronounced with the use of the preferred salts of the acid, potassium hydroxycitrate and potassium-magnesium hydroxycitrate, and may be further potentiated by the use of a controlled-release form of the compound. The discovery that HCA has ACE/renin-angiotensin-aldosterone system-regulating effects allows for the creation of novel and more efficacious approaches to preventing and ameliorating diseases and other conditions. Furthermore, this discovery makes possible the development of adjuvant modalities that can be used to improve the results realized with other treatment compounds while at the same time reducing the side effects normally found with such drugs. HCA delivered in the form of its potassium salt is efficacious at a daily dosage of between 750 mg and 10 grams, preferably at a dosage of between 3 and 6 grams for most individuals with the dosage divided into two or three deliveries. A daily dosage above 10 grams might prove desirable under some circumstances, such as with extremely large or resistant individuals, but this level of intake is not deemed necessary under normal conditions.

### Objects and Advantages

It is an objective of the present invention to provide a method for preventing, treating or ameliorating conditions that involve the metabolism of the angiotensin-converting enzyme (ACE) and the renin-angiotensin-aldosterone system. It is a further object of the present invention to provide a means of treating or ameliorating conditions that arise from excessive ACE activity. These include cardiovascular diseases in general, heart failure, ventricular remodeling, ejection fraction issues, atrial fibrillation, and a wide variety of renal conditions. It is yet a further advantage of the present invention to provide a means—one which is accompanied by few or no side effects—of maintaining such improved status without resort to special diets. Knowledge of the present invention has the advantage of allowing the use of forms of (−)-hydroxycitric acid, including especially through controlled release formulations, as adjuvants to cardiovascular drugs and to drugs designed to stabilize or improve renal health. Other health conditions discovered to be influenced by the angiotensin-converting enzyme (ACE)/renin-angiotensin-aldosterone system would similarly be expected to be influenced.

### DESCRIPTION OF THE PREFERRED EMBODIMENTS

The free acid form and various salts of (−)-hydroxycitric acid (calcium, magnesium, potassium, sodium and mixtures of these) have been available commercially for several years. Any of these materials can be used to fulfill the invention revealed here, but with varying degrees of success. These materials are generally useful in this descending order of efficacy: potassium salt, sodium salt, free acid, magnesium salt, and calcium salt. Exact dosing will depend upon the form of HCA used, the weight of the individual involved, and the other components of the diet. Controlled release can also be expected to improve results by aiding in maintaining a sustained exposure to the drug as required for therapy. The previously patented hydroxycitric acid derivatives (mostly amides and esters of hydroxycititric acid, the patents for which are now expired, to wit, U.S. Pat. Nos. 3,993,668; 3,919,254; and 3,767,678) likely are roughly equivalent to the HCA sodium salt in efficacy.

### EXAMPLE 1

### Evidence From Blood Pressure Modulation

A know effect of ACE inhibitors is a reduction in elevated systolic blood pressure. To test this, the following protocol was employed: Sprague-Dawley Rats (SD), approximately 8 weeks of age werw obtained. Six groups of eight male SD received the same standard rat chow manufactured to specifications. The special diets derived 30% of calories from fats (one half from lard and one half from corn oil), 50% from carbohydrates, and 20% from proteins. Twenty percent of dietary calories was derived from sucrose and the preponderance of the remaining carbohydrate calories was derived from dextrin. During weekdays (M-F), each group was gavaged twice daily with a solution containing a commercial source of potassium hydroxycitrate (KHCA), a commercial source of potassium-calcium hydroxycitrate (KCaHCA), or a pre-commercial non-salt source of potassium-magnesium hydroxycitrate (KMgHCA, listed as KMgHCA L-Low, M-Intermediate or H-High depending upon the dose). Over the weekends (S-S), a similar quantity of the weekday daily dose was added to twenty grams of food, that is, an amount of food estimated to be close to the daily intake of the animals. At initiation of study and four weeks, and eight weeks later, bloods were drawn from all SD for routine blood chemistries. Body weight (BW) was measured weekly and systolic blood pressure (SBP) was measured every two weeks.

The HCA dosages in the arms varied. The dosage used in the KHCA arm was extrapolated from the recommended 1,500 mg HCA per day for humans consuming a normal diet (i.e., ≧30% calories derived from fats) advocated by a commercial seller of KHCA and claimed to have produced acceptable clinical results. The approximate equivalent for the rat model is 35.4 mg HCA per day, which we increased to 38.4 mg HCA per day for convenience in employing a 48% HCA potassium salt and to remain safely on the high side in practice. For the sake of comparison, a commercial KCaHCA salt (60% HCA) was chosen and delivered at an HCA dosage level of 48 mg per day, which slightly exceeded the lowest dosage of HCA found to be efficacious for inhibition of weight gain in rats in the early pharmaceutical trials (45.4 mg/day) using pure trisodium hydroxycitrate and a very low fat diet. The design thus utilized a realistic diet with rough equivalents of the HCA dosages claimed to be effective in both the human and rat models.

Calculations were based on the early work on HCA by Roche in which the lowest dose in rats shown to be efficacious in reducing weight gain was 0.33 mmol/kg twice a day (delivered as trisodium hydroxycitrate) on a diet consisting of 70% glucose and 1% fat [8]. (−)-Hydroxycitric acid (C₆H₈O₈) has a molecular weight of 208, therefore 1 millimole=208 mg. The rat dose thus would be calculated as 0.33 mmol/kg b.i.d., meaning 208×0.33 kg rat wt (in kg assuming an average weight of 333 grams)=22.65/1000=22.7 mg b.i.d. or 45.4 mg HCA total intake per day, which is equivalent to 76 mg daily of a 60% HCA salt. This should be put in perspective as to the likely lowest efficacious human dose under similar conditions of less than 10% calories from fat in the diet. At 0.33 mmol HCA b.i.d., the human dosage is 208 mg×0.33×70 kg=4.8 grams of HCA per dose×2=9.6 grams HCA/day=16 grams of a 60% salt. Using the normal rat-to-human multiplier for calculating the small animal effect [9], an appropriate dose for humans would be close to 9.6÷5=1.92 grams hydroxycitric acid content on an extremely low fat diet and assuming the material is supplied via a salt that is equivalent to pure trisodium hydroxycitrate in efficacy and is delivered without food effect on uptake.

The experimental KMgHCA dosings varied considerably from that of the other two salts. Subsequent to the start of the trials, it was discovered that the KMgHCA was diluted with as much as 15% potassium chloride (inactive) and that there was a mistake in the calculation of the waters of hydration. As a result, the recalculated HCA doses for the experimental compound were a low dose (KMgHCA L) of 14 mg, an intermediate dose (KMgHCA M) of 28 mg and a high dose (KMgHCA H) of 84 mg per day. The difficulty in calculating the HCA content in this case is not unique inasmuch as there is as of yet no universally accepted method for calculating the HCA content of the various salts. Again, preparations yielded the equivalent of 48 mg HCA per day from KCaHCA and 38.4 mg HCA per day from KHCA.

Systolic Blood Pressure (SBP): SBP was estimated by tail plethysmography in unanesthetized rats after a brief warming period. Readings were taken approximately one minute apart. To be accepted, SBP measurements had to be virtually stable for a minimum of three consecutive readings.

Statistical Analyses: Results are presented as mean±SEM. Many statistics were performed by one-way analysis of variance (ANOVA). SBP and BW were examined by two-way analyses of variance (one factor being dietary group and the second factor being time of examination). Where a significant effect of diet was detected by ANOVA (p<0.05), the Dunnett t test was used to establish which differences between means reached statistical significance (p<0.05). If a Student's t test was employed, this is noted.

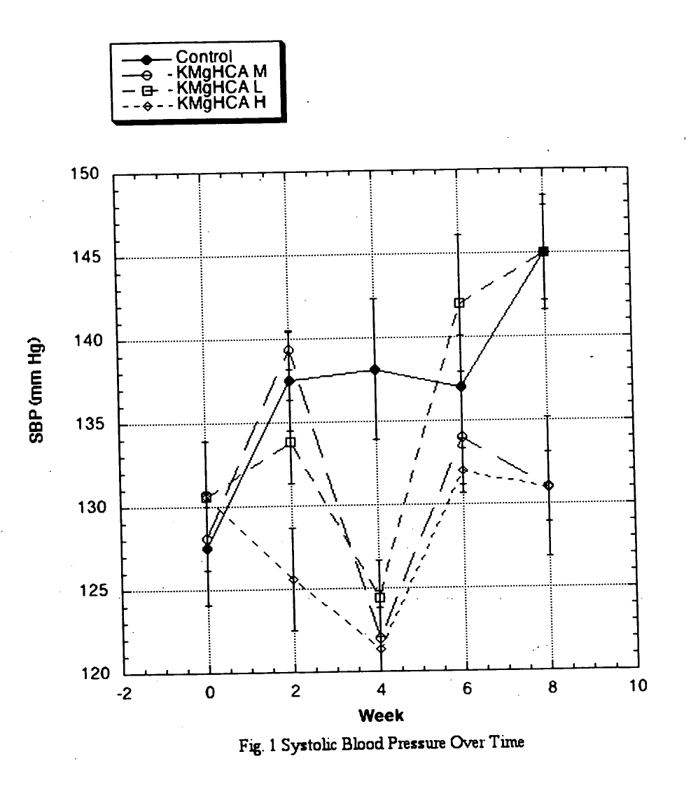

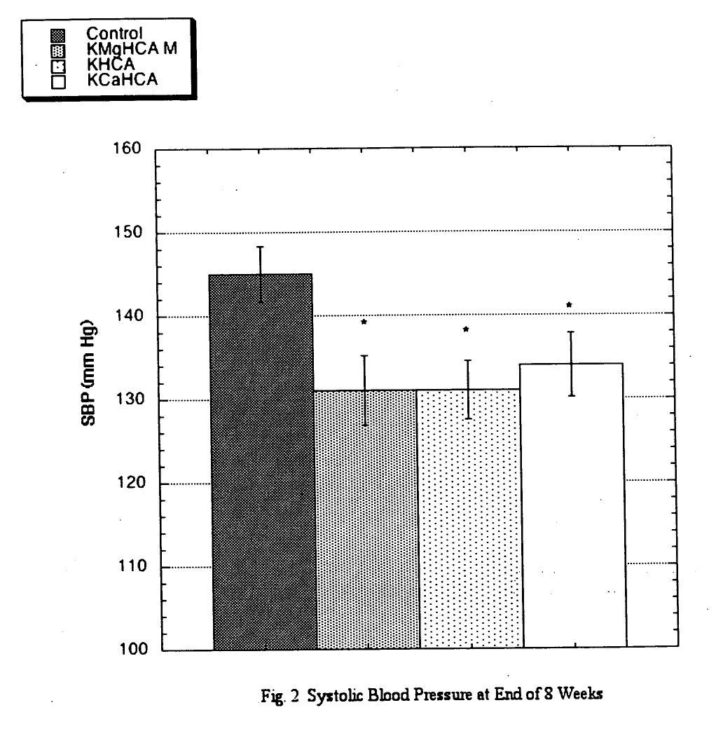

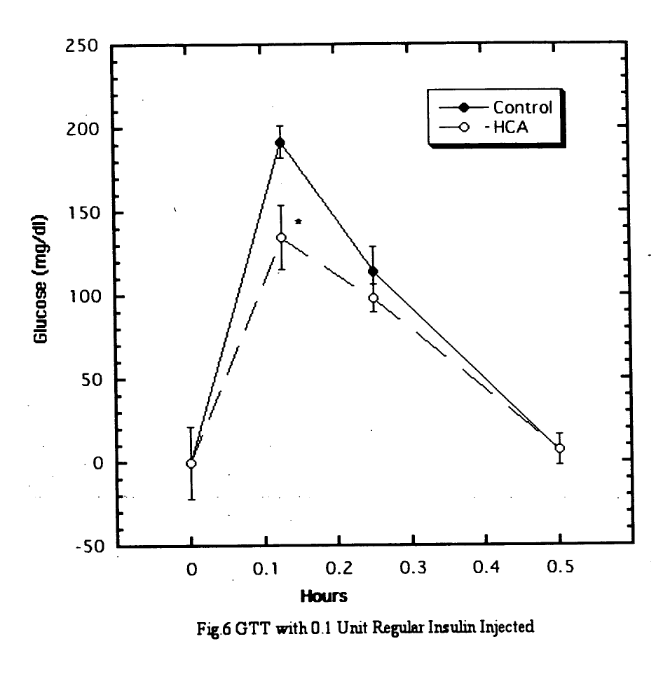

Findings for Systolic Blood Pressure: The general trend was for all test groups to consistently show significantly lower SBP during the course of study. The only exception was low-dose of KMgHCA (KMgHCA L), which apparently was below the threshold for effect (FIG. 1). At the end of eight weeks, the doses of the KHCA and KCaHCA and the two higher doses of the KMgHCA caused significant decreases in SBP compared to control (FIG. 2). With regard to 3 different doses of KMgHCA (FIG. 6), the low dose essentially did nothing, but the intermediate and high doses caused virtually the same significant lowering of SBP at the end of 8 weeks—over 10 mm Hg.

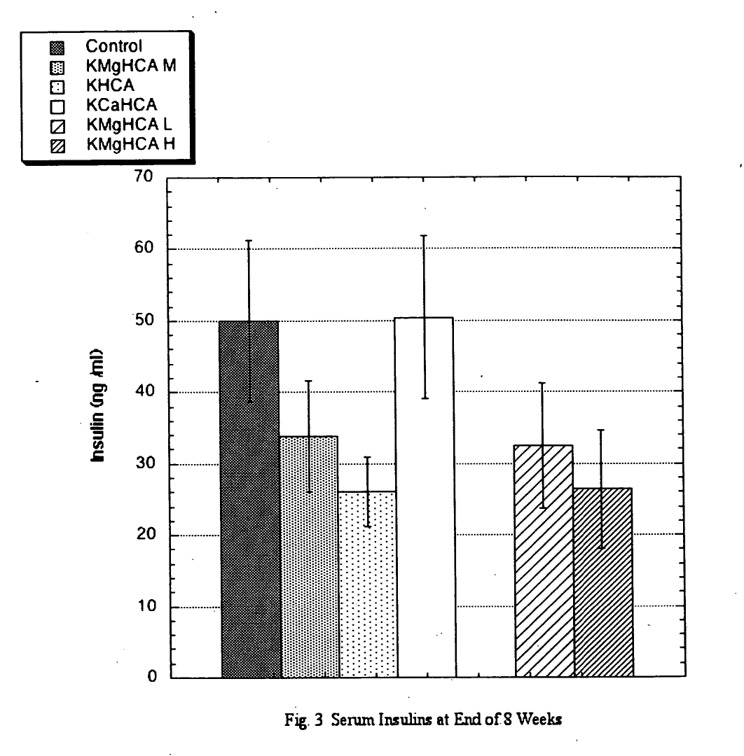

Findings for Blood Chemistries: Blood chemistries were obtained at baseline, one month and two months. No significant differences were seen in BUN, and serum creatinine, ALT, AST, and glucose among the six groups. Accordingly, no evidence of liver and renal toxicities was apparent. Although the average insulin concentrations were lower in all KMgHCA groups and in the KHCA group (FIG. 3), the differences were not significant compared to control using ANOVA. The lack of significance may be due to the small numbers of animals examined and the large variances found, especially with control. Only the KCaHCA group did not show a trend toward lower circulating insulin. Recalculating control versus KHCA alone for insulin using the Student's t test showed significance; a similar recalculation of control versus KMgHCA H was at the margin of significance (p=0.058).

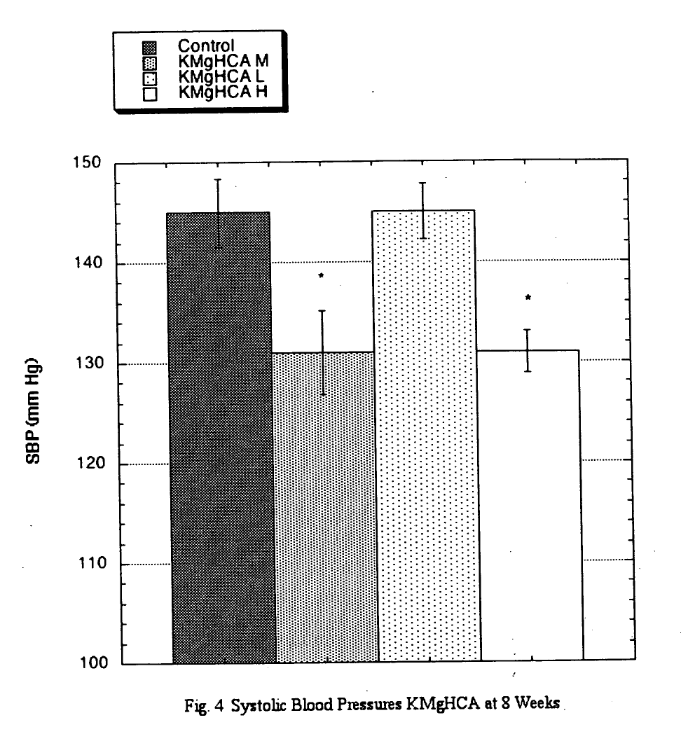

An earlier study not described here had demonstrated a decrease in SBP using a KCaHCA salt at a dose of 120 mg HCA per day. In the present study, significantly decreased SBP was produced readily in all the hydroxycitrate groups with the exception of the low dose of KMgHCA (14 mg HCA). One surprising finding was that that the intermediate dose of KMgHCA supplying only 28 mg HCA (KMgHCA M) was equal in this regard to KHCA supplying 38.4 mg HCA and KCaHCA supplying 48 mg HCA (FIG. 2). Another interesting outcome was that elevating the dose of HCA further, in this case to 84 mg in the high KMgHCA dose (KMgHCA H) did not have exert a greater impact on SBP (FIG. 4). Taken together, these findings suggest that there may be a limit to the blood pressure effect of HCA and that this limit is reached with a relatively low dose. Whether all the salts are equally effective remains to be seen. With regard to at least one of the vectors influencing blood pressure, insulin, the KCaHCA salt appears to be significantly less active than the others tested. Moreover, the fact that KCaHCA had little positive impact upon insulin regulation in this model, yet still improved SBP suggests that more than one blood pressure regulating mechanism is at work.

### EXAMPLE 2

### Response to Losarten Challenge

Many factors can positively influence blood pressure, e.g., diuretics, antioxidants, regulators of sympathetic/parasympathetic tone, compounds that improve insulin sensitivity and so forth. Therefore, losartan, an angiotensin-2 receptor blocker, was utilized to discover whether the ACE system was involved in the results discussed in Example 1.

Spontaneously hypertensive rats (SHR) were placed on a diet composed of regular rat chow (60% w/w) and table sugar (40% w/w). This diet reliably elevates blood pressure in this animal model. One group received 100 mg HCA per day in the form of a new potassium-magnesium hydroxycitrate (different from that used in Example 1) via an added 5 g HCA per kg of food mix. Systolic blood pressure and body weight were tested as in Example 1 on a weekly basis.

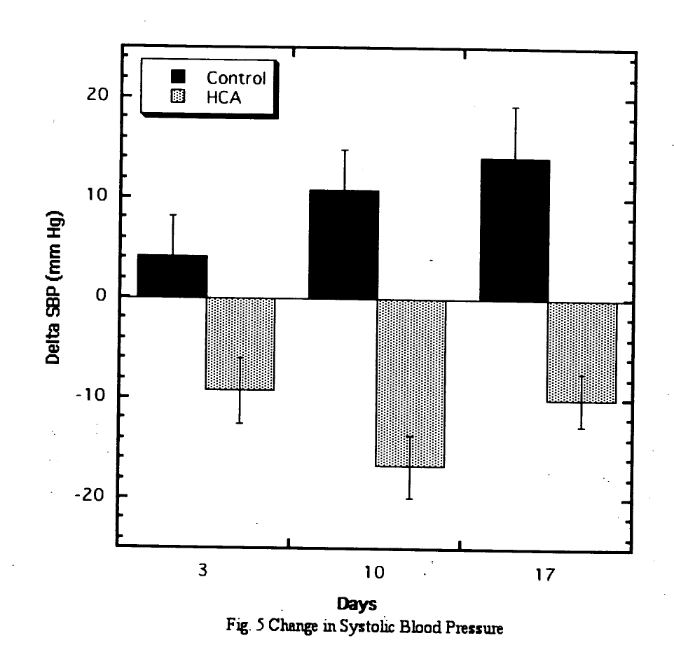

Over three weeks, there was a trend for an increase in body weight in SHR consuming KMgHCA (p=0.084) in this model. This was viewed as likely positive in that rats gain weight steadily as long as they remain in good health and the SHR at middle age, as used here, lives a relatively short life and its health deteriorates as its blood pressure rises. SBP steadily increased in control as shown in FIG. 5, where delta SBP steadily increased in control. In contrast, the KMgHCA rats showed a decrease in SBP from baseline. A glucose tolerance test was administered in which 0.1 unit of regular insulin was injected along with glucose. At 7.5 minutes, there was a significantly lesser rise in glucose appearance in bloodstream. This finding indicates increased insulin sensitivity. (FIG. 6)

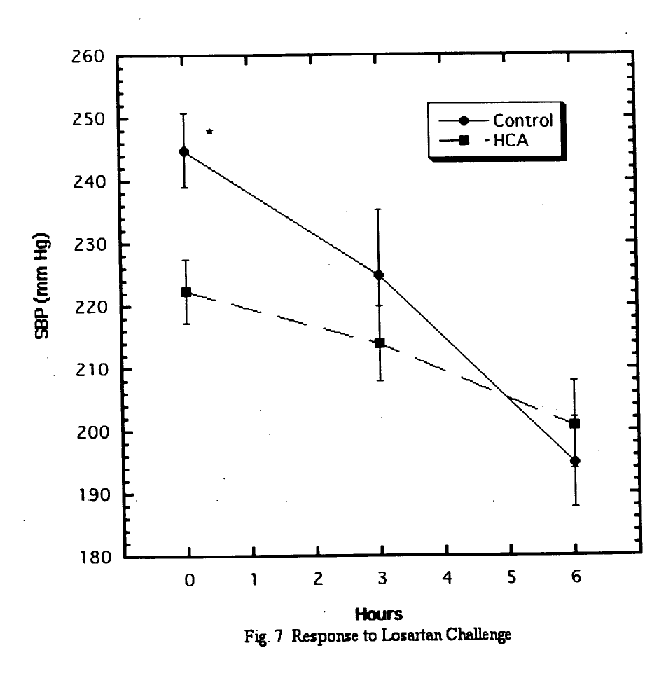

When losartan was injected, the SBP of both groups decreased. At 6 hours, the SBP were essentially the same. As shown in FIG. 7, the decreases in SBP's at 6 hours (−50±16.1 vs −21.7±7.0) were significantly different (p=0047). Thus, HCA appears to decrease angiotensin-2 in rats and to lower elevated SBP. Although insulin regulation likely is a factor in the blood pressure modulating effect of HCA, this evidence argues that inhibition of ACE is also important. Moreover, taken together with the evidence in Example 1, this second experiment helps to explain the difference in efficacy in blood pressure regulation between KCaHCA and the other HCA salts tested, to wit, although KCaHCA has little impact upon insulin metabolism, it nevertheless moderates blood pressure via ACE inhibition. Thus there is both direct and indirect evidence from experiments with several different salts of HCA indicating that the compound modulates ACE metabolism.

### EXAMPLE 3

### A Standard Dosage Form

Numerous methods can be given as means of delivering HCA as required by the invention, including capsules, tablets, powders and liquid drinks. The following preparation will provide a stable and convenient dosage form.

| | | | 1 Kg |
|---|---|---|---|
| Ingredient | Weight | Percent | Batch |
| 1. Aqueous Potassium Hydroxycitrate | 500 gm | 62.5% | 0.63 |
| 2. Calcium Carbonate | 50 gm | 6.25% | 0.06 |
| 3. Potassium Carbonate | 50 gm | 6.25% | 0.06 |
| 4. Anhydrous Lactose | 150 gm | 18.75% | 0.19 |
| 5. Cellulose Acetate Pthalate Acetate | 50 gm | 6.25% | 0.06 |
| Total | 800 gm | 100.00% | 100.00 |

A. Blend items 1-5 in mixing bowl until smooth and even.
B. Take the liquid and spray into spray-drying oven at 300° C. until white powder forms. When powder has formed, blend with suitable bulking agent, if necessary, and compress into 800 mg tablets with hardness of 10-15 kg. This will mean that each tablet, if starting with 62% KHCA polymer powder, will have about 31% KHCA. However, if the tablets are pressed to 1600 mg, the dose will be equal to 800×62% KHCA.
C. After pressing the granulate through the screen, make sure that it flows well and compress into oblong tablets.
D. Tablets should have a weight of 1600 mg and a hardness of 14±3 kg fracture force. When tablets are completed, check for disintegration in pH 6.8, 0.05M KH2PO4. Disintegration should occur slowly over 4-5 hours.

### EXAMPLE 4

### An Enteric Softgel Dosage Form

Soft gelatin encapsulation is used for oral administration of drugs in liquid form. For this purpose, HCA may be provided in a liquid form by suspending it in oils, polyethylene glycol-400, other polyethylene glycols, poloxamers, glycol esters, and acetylated monoglycerides of various molecular weights adjusted such as to insure homogeneity of the capsule contents throughout the batch and to insure good flow characteristics of the liquid during encapsulation. The soft gelatin shell used to encapsulate the HCA suspension is formulated to impart enteric characteristics to the capsule to ensure that the capsule does not disintegrate until it has reached the small intestine. The basic ingredients of the shell are gelatin, one or more of the enteric materials listed above, plasticizer, and water. Care must be exercised in the case of softgels to use the less hygroscopic salts and forms of HCA or to pretreat the more hygroscopic salts to reduce this characteristic. The carrier may need to be adjusted depending on the HCA salt, ester or amide used so as to avoid binding of the ingredients to the carrier. Water should never be used as a carrier. Various amounts of one or more plasticizer are added to obtain the desired degree of plasticity and to prevent the shell from becoming too brittle.

### EXAMPLE 5

### A Controlled-delivery Dosage Form

| Ingredient | mg/Tablet | Percent |
|---|---|---|
| 1. HCA calcium salt | 500.00 mg | 71.43% |
| 2. Microcrystalline cellulose | 17.00 mg | 2.42% |
| 3. Dicalcium phosphate | 45.00 mg | 6.42% |
| 4. Corn starch | 9.00 mg | 1.28% |
| 5. TPGS | 46.00 mg | 6.60% |
| 6. Hydrogenated vegetable oil | 50.00 mg | 7.14% |
| 7. Cellulose acetate phthalate | 15.00 mg | 2.14% |
| 8. Carbopol ® 974P Carbomer | 15.00 mg | 2.14% |
| 9. Magnesium Sterate | 3.00 mg | 0.43% |
| TOTAL | 700.00 mg | 100.00% |

1. Weigh and blend items 1-4 in a fluid bed dryer and blend for 4-5 minutes. Dissolve item #5 by heating to 40° C. until molten then stir with magnetic stir rod. After the powders are blended, continue steady blending while adding the TPGS as a molten liquid. Pour in all fluid until an even granulate is formed. Next melt the hydrogenated vegetable oil until molten and fluid in nature. Spray this material at the same time stirring with a magnetic stir rod. Continue blending with air at 30° C. When all the material is thoroughly coated and the granulate is hardened, spray the cellulose acetate phthalate which has been completely dissolved in ammoniated water. Continue spraying until all the granulate has been covered then allow to dry at room temperature in the fluid bed dryer with continuous blending. Remove the granulate from the bowl, when the granulate is dry, pass through an #093 screen using a D3 Fitzmill comminutor.
2. When the granulate has been dried and reduced in size, blend in fluid bed first with Carbopol 974P, then when completely blended, add magnesium stearate and blend for 2-3 minutes.
3. Place the mixed granulate on a rotary press and compress the material into tablets with a weight of 700 mg and a fracture force of 10-15 kg.

### CONCLUSIONS

(−)-Hydroxycitrate has a multitude of metabolic functions. The literature teaches that the compound reduces blood lipids, induces weight loss and decreases appetite in both animals and humans. However, the inventor has discovered that this compound can be employed to positively influence the angiotensin-converting enzyme (ACE)/renin-angiotensin-aldosterone system. This safe and effective amelioration of ACE-related problems is an entirely unexpected and novel use of (−)-hydroxycitric acid, its derivatives and its salt forms.

I claim:

1. A method for preventing, treating or ameliorating angiotensin-converting enzyme (ACE)/renin-angiotensin-aldosterone system related symptoms in an individual in need thereof which is comprised of administering orally an effective amount of (−)-hydroxycitric acid.

2. The method of claim 1 where the (−)-hydroxycitric acid is supplied in a therapeutically effective amount of the free acid or its lactone.

3. The method of claim 1 where the (−)-hydroxycitric acid is supplied in a therapeutically effective amount of the alkali metal salts potassium or sodium (−)-hydroxycitrate.

4. The method of claim 1 where the (−)-hydroxycitric acid is supplied in a therapeutically effective amount of the alkaline earth metal salts calcium or magnesium (−)-hydroxycitrate.

5. The method of claim 1 where the (−)-hydroxycitric acid is supplied in a therapeutically effective amount of a mixture the alkali metal salts and/or the alkaline earth metal salts of (−)-hydroxycitrate or some mixture of alkali metal salts and alkaline earth metal salts of (−)-hydroxycitrate or in the form of therapeutically effective amide and/or ester derivatives of (−)-hydroxycitric acid.

6. The method of claim 1 where the (−)-hydroxycitric acid is supplied in a therapeutically effective amount as the free acid, its lactone or as one or more of the salts or other derivatives of the free acid and is delivered in a controlled release form.
